(19) United States
(12) Patent Application Publication
(10) Pub. No.: US 2004/0106104 A1
(43) Pub. Date: Jun. 3, 2004

(54) Viral envelope mediated fusion assay

(76) Inventors: Pin-Fang Lin, Long Valley, NJ (US); Carol Deminie, Middletown, CT (US); Robert A. Fridell, Guilford, CT (US); Brett Robinson, Preston, CT (US); Ronald E. Rose, Madison, CT (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/459,069

(22) Filed: Jun. 11, 2003

Related U.S. Application Data
(60) Provisional application No. 60/388,077, filed on Jun. 11, 2002.

(51) Int. Cl. C12Q 1/70; C12Q 1/68

(52) U.S. Cl. 435/5; 435/6

(57) ABSTRACT

This invention provides a method of screening a sample for susceptibility to a viral entry inhibitor. The invention further provides a method for monitoring a patient for sensitivity to a viral entry inhibitor and a method of determining an inhibitory concentration of a viral entry inhibitor to inhibit virus infection in a patient. A method of screening for viral entry inhibitors is also provided.

This application claims the benefit of provisional U.S. Application Ser. No. 60/388,077, filed Jun. 11, 2002, which is incorporated herein by reference in its entirety.

### FIELD OF THE INVENTION

The present invention relates to a viral envelope mediated fusion assay for determining the susceptibility of viruses to viral entry inhibitors and identifying viral entry inhibitors. More particularly, this invention relates to monitoring sensitivity of clinical samples from virally infected patients to viral entry inhibitors.

### BACKGROUND OF THE INVENTION

Viruses such as human immunodeficiency virus (HIV) have high replication and mutation rates that result in a diverse population of viruses (quasi species) within an infected individual. For example, the sequence diversity in the human immunodeficiency virus type 1 (HIV-1) envelope gene within infected individuals has been estimated to be as high as 10% (Anderson et al., J. Mol. Evol., 53: 55-62 (2001)). This diversity poses a challenge to the development of molecular methods to monitor sensitivity of a patient to antiviral therapies, especially, antiviral agents that block the binding of viruses to host cells.

The entry of viruses such as HIV-1 into host cells is initiated by the binding of the virus to the host cell surface. This binding is accomplished through specific interactions between the virus envelope proteins and host cell receptor proteins. For HIV-1, the viral envelope glycoprotein, gp120, binds to the T-cell receptor CD4 and a coreceptor, particularly a chemokine receptor, and more particularly either CXCR4 (X4-specific viruses) or CCR5 (R5-specific-viruses). T-tropic strains of HIV use the (α-chemokine receptor CXCR4 for viral entry while M-tropic strains of HIV use the β-chemokine receptor CCR5 for viral entry. Dual-tropic strains of HIV are capable of using either the CXCR4 or CCR5 receptor for viral entry. Other chemokine receptors, for example, CCR2b and CCR3 may also be utilized by the virus to enter a host cell. These binding events induce the fusion of the viral membrane with the cell membrane permitting the viral genome to enter the host cell. Once inside the cell, the viral genome establishes an infection and directs the host cell to produce progeny virions. It is desirable to identify antiviral agents that target the entry of the virus into host cells by blocking the interaction of the viral envelope protein with one or more of the host cell receptor proteins. For example, HIV entry inhibitors interfere with or inhibit the ability of the HIV gp120 envelope protein to bind to the CD4 receptor and/or one or more chemokine receptors.

A major limitation of known methods for monitoring sensitivity of viruses to viral entry inhibitors, particularly HIV entry inhibitors, is the difficulty in cloning functional envelope genes from clinical samples. This is due in part to the poor cloning efficiency and instability of the viral envelope sequence in host cells such as E. coli. The cloned envelopes often contain frame shift or stop codon mutations that prevent the expression of full length envelope proteins. Viral proteins of interest obtained by traditional cloning methods are utilized in assays to identify antiviral agents. Traditional methods for cloning of viral DNA involve a number of steps performed over a period of days to obtain only one particular nucleotide sequence encoding a viral protein of interest. However, in those traditional cloning methods, the particular cloned nucleotide sequence is obtained from a sample that contains a diverse population of genes encoding the viral protein. Thus, many viral proteins are unlikely to be represented in the antiviral screening assays. Conventional cloning steps involve ligation of a vector and a selected nucleic acid sequence, and transformation of host cell with the vector. Individual colonies must then be selected and analyzed to obtain the desired clone. DNA from the selected colony must then be purified prior to being used for a functional assay. A major drawback of this conventional cloning approach, especially with respect to HIV-1 envelope proteins, is that the resulting clone is derived from a single nucleotide sequence and does not represent the diversity of the viral sequences present in a given patient. One approach to avoid this lack of representation is to obtain large numbers of clones that are pooled for use in a sensitivity assay so that the diversity of the viral genome is more accurately represented. However, the need to obtain large numbers of clones requires additional time and effort. In addition to being cumbersome and time consuming, traditional cloning methods are also subject to difficulties inherent in cloning functional viral envelope genes in traditionally used host cells such as E. coli.

Given the genetic diversity of virus envelope glycoproteins and the presence of viral quasi species in an infected individual, a need exists for rapid, efficient and accurate methods to monitor the sensitivity of patient viruses to viral entry inhibitor compounds. More particularly, there is a need for methods for monitoring the development of clinical resistance to viral entry inhibitors, especially HIV entry inhibitors. Moreover, there is a need for a method of monitoring sensitivity of viruses to viral entry inhibitor compounds that accounts for diversity in the viral envelope proteins, especially HIV envelope proteins, in a given individual and the human population.

### SUMMARY OF THE INVENTION

The present invention provides a rapid and sensitive assay for determining the susceptibility of viral laboratory and clinical samples to viral entry inhibitors. In accordance with the present invention, the sensitivity of a virus to viral entry inhibitors is determined using a viral envelope mediated cell fusion assay as shown in FIG. 1. The viral envelope mediated cell fusion assay of the present invention utilizes expression of viral envelope proteins directly from a PCR fragment without prior cloning of the viral envelope. The invention described herein does not require infectious virus but rather, employs a linear viral envelope expression element. The problematic viral envelope cloning step required for envelope expression of known viral envelope assays has been replaced with a technique that allows direct expression of linear viral envelope PCR fragments. This novel approach more accurately represents the diversity of envelope genes found in virus infected clinical samples.

Thus, the present invention provides an assay to evaluate the potency or efficacy of viral entry inhibitors against diverse viral envelope genes found in infected clinical samples. The assay of the present invention is particularly useful for identifying HIV entry inhibitors and monitoring the development of resistance of HIV to HIV entry inhibitors.

The envelope mediated cell fusion assay of this invention utilizes effector cells expressing linear viral envelope expression elements and target cells expressing receptors necessary for binding and fusion of the target cell with an effector cell. In accordance with this invention, an envelope gene from virus infected samples is PCR amplified and directly transfected into an effector cell. The effector cells transiently express viral envelope protein and a transcription factor. The target cells contain a reporter gene driven by a promoter which is responsive to the transcription factor in the effector cells. Mixing of the target cells and effector cells results in cell-to-cell fusion that triggers activation of the target cell reporter gene by the effector cell transcription factor. Production of the reporter protein is monitored to determine sensitivity of the viral envelope to viral entry inhibitors.

This invention further provides a method for monitoring the susceptibility of a patient to a viral entry inhibitor. The invention further provides a method of determining an inhibitory concentration of a viral entry inhibitor to inhibit virus infection in a patient. A method for screening for viral entry inhibitors is also provided.

The methods of the invention may be performed manually. Preferably, the methods of the invention are performed using an automated system to achieve high-throughput.

### BRIEF DESCRIPTION OF THE FIGURES

FIG. 1 is a schematic respresentation of the viral envelope mediated cell fusion assay of the present invention.

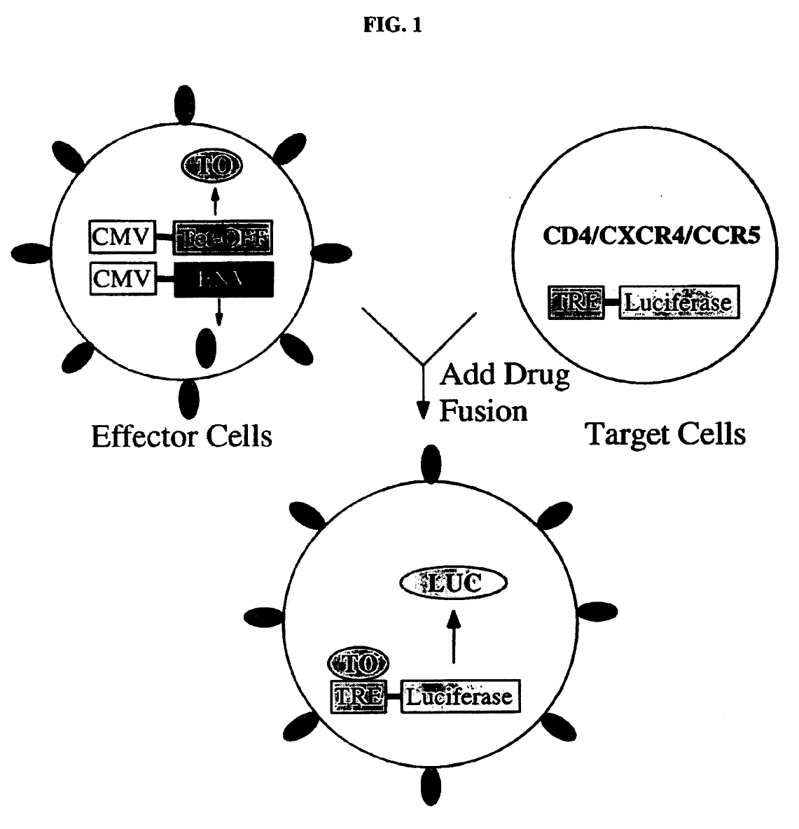

FIG. 2 is a schematic representation of a method for making a linear HIV envelope expression element.

FIG. 3 is a schematic representation of the viral envelope mediated fusion assay of the present invention wherein the effector is a pseudovirus.

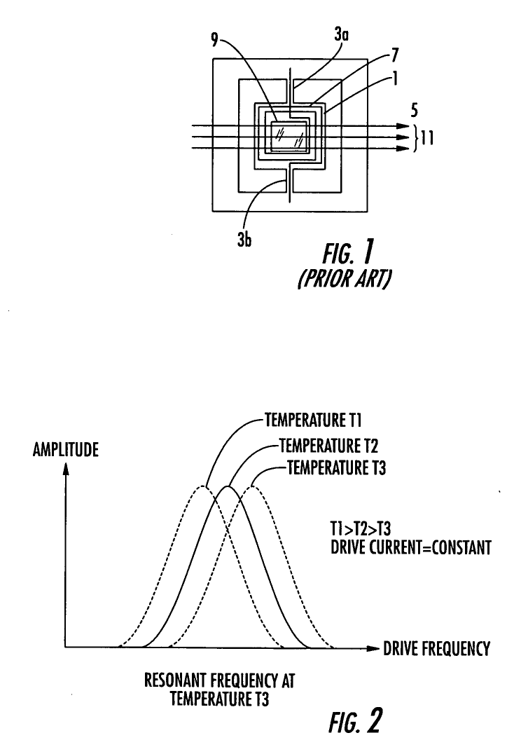

### DETAILED DESCRIPTION OF THE INVENTION

The present invention provides a rapid and comprehensive assay to determine the sensitivity of viral samples to viral entry inhibitors. The assay of the present invention utilizes linear viral envelope expression elements produced by PCR and allows for a population of envelope proteins from a clinical sample to be efficiently expressed. The linear viral envelope expression elements are representative of the genetic variability of nucleic acids encoding viral envelope proteins. In accordance with this invention, novel viral entry inhibitors can be identified and the efficacy of known viral entry inhibitors can be evaluated. The assay of the present invention is particularly useful for determining sensitivity of HIV to HIV entry inhibitors. The assay can also be used to determine the sensitivity of other viruses to viral entry inhibitors such as, for example, herpes simplex virus (HSV), varicella zoster virus (VSV), cytomegalovirus (CMV), Epstein-Barr Virus (EBV) and other human viruses.

As used herein, the “sensitivity” of a virus or patient infected with the virus to a viral entry inhibitor is the ability of a viral entry inhibitor to cause some measurable amount of inhibition of viral replication. The term “susceptibility” is used herein interchangeably with the term “sensitivity.” The term “inhibitory concentration” as used herein is intended to mean the concentration of a viral entry inhibitor at which the compound inhibits a measurable percentage of virus replication. The term “EC50” as used herein is the concentration of the viral entry inhibitor at which 50% of viral replication is inhibited. Examples of inhibitory concentration values are EC50, EC60, EC70, EC80 and EC90 which represent the concentration of viral entry inhibitor at which 50%, 60%, 70%, 80% and 90% of virus replication is inhibited, respectively.

The term “resistance” as used herein refers to natural or acquired decreased sensitivity of a virus or patient infected with a virus to a viral entry inhibitor. “Viral entry inhibitors” referred to herein can be any compound capable of interfering with the entry of viruses into cells. The term “compound” as used herein includes but is not limited to small molecules, peptides, nucleic acid molecules and antibodies.

The assay of the present invention is particularly useful for evaluating the sensitivity of HIV to HIV entry inhibitors, monitoring development of resistance to HIV entry inhibitors and identifying HIV entry inhibitors. In a preferred embodiment, the present invention is used to evaluate sensitivity or resistance of HIV-1 to HIV entry inhibitors and identify HIV-1 entry inhibitors.

In accordance with the present invention, functional viral envelopes are provided by directly expressing transcriptionally active PCR amplified linear viral envelope expression elements in effector cells which eliminates the need for cloning of HIV envelope sequences. In accordance with the present invention, elimination of the cloning step permits the expression of functional envelope genes that better represent the diversity of envelope genes in a particular HIV clinical sample and the human population. This approach allows the diversity of the envelope genes found in HIV infected clinical samples to be more accurately represented. As used herein, the term “linear viral envelope expression element” refers to a linear nucleic acid construct encoding a viral envelope protein or a portion thereof and having a 5′ element and 3′ element which allows for expression of the linear viral envelope expression element in a host cell. In accordance with the invention, both functional and non-functional envelope sequences in the viral population from viral clinical samples are PCR amplified. Therefore, the 5′ and 3′ elements can be added to both functional and non-functional envelope sequences. Preferably, the 5′ and 3′ elements also include nucleic acid sequences that can be used as primers to further amplify the linear viral envelope expression element.

The term “linear HIV envelope expression element” as used herein refers to a linear nucleic acid construct encoding an HIV envelope protein or a portion thereof and having a 5′ element and 3′ element for expression of the HIV envelope protein in a host cell.

The linear viral envelope expression elements can be prepared using an enzyme such as vaccinia topoisomerase I (Shuman S., Proc. Natl. Acad. Sci. USA, 88: 10104-10108 (1991) and Shuman, S., J.Biol. Chem., 269: 32678-32684 (1994)) capable of joining PCR products to selected 5′ and 3′ sequences for expression of the PCR fragment in a host cell. The double stranded PCR product including the selected 5′ and 3′ sequences is then used as a template to generate additional copies of the linear viral envelope expression element. The advantage of this technique is that PCR generated nucleic acid sequences encoding a viral envelope can be directly introduced into a host cell without the need for additional cloning steps.

In a preferred embodiment of the present invention, the nucleic acid sequence encoding the viral envelope protein is obtained by amplifying a viral envelope gene of interest. Conventional techniques known to those skilled in the art can be used for amplifying a nucleic acid sequence encoding a viral envelope protein or portion thereof. Preferably, amplification of the viral envelope gene is accomplished by PCR, and more preferably by reverse transcription PCR. The envelope gene can also be PCR amplified from infected cells that have the viral genome integrated into the chromosomal DNA. In one embodiment of the present invention, PCR primers containing base pair sequences that facilitate joining of 5′ and 3′ elements for protein expression are utilized to amplify the nucleic acid sequence of interest and produce the desired viral envelope PCR fragment. Examples of commercially available methods for generating transcriptionally active nucleic acid fragments are the TOPO Tools Technique from Invitrogen Corporation, Carlsbad, Calif., and the TAP Express Rapid PCR Expression System from Gene Therapy Systems, San Diego, Calif. The TOPO Tools technology is based on directional topoisomerase-I-mediated joining of double-strand DNA as described by Cheng, C., and Shuman, S., Mol. Cell. Biol., 20: 8059-8068.

In accordance with a preferred embodiment of the present invention, an HIV envelope gene can be PCR amplified using the following primers: BF 17 5′-ATGGAGCCAGTAGATCCTAGACTAGAGCCCTGG-3′ (SEQ ID NO: 1); and ENV-N 5′-CTGCCAATCAGGGAAGTAGCCTTGTGT-3′ (SEQ ID NO: 2). The PCR product is then purified and an additional set of primers is used to introduce the proper ends for the TOPO reaction.

In the most preferred embodiment of the present invention, forward and reverse primers having an 11 base pair sequence including (i) a six base pair sequence which is capable of binding to the overhang sequence on the TOPO 5′ or 3′ element to facilitate directional joining of the 5′ and 3′ elements and (ii) a five base pair sequence which is complementary to the recognition site (CCCTT) of topoisomerase I are utilized to generate PCR fragments of the viral gene of interest. Preferably, the following primers are used to generate PCR fragments of viral envelope protein by the TOPO technique:

Forward primer: 5′-CGGAACAAGGG-3′ (SEQ ID NO: 3)

Reverse primer: 5′-TGAGTCAAGGG-3′ (SEQ ID NO: 4) Particular examples of primers that can be used to produce linear HIV envelope expression elements are the following: BR1072 5′-CGGAACAAGGGCTTAGGCATCTCCTATGGCAGGAAGAA-3′ (SEQ ID NO: 5); and BR1075 5′-TGAGTCAAGGGTAGCCCTTCCAGTCCCCCCTTTTCTTTTA-3 (SEQ ID NO: 6); or CDIN5′ 5′-CGGAACAAGGGATGGCAGGAAGAAGAAGCGGAGACAGC-3′ (SEQ ID NO: 7); and CDIN3′ 5′-TGAGTCAAGGGTCTTATAGCAAAATCCTTTCCAAGCCCTGT-3′. (SEQ ID NO: 8).

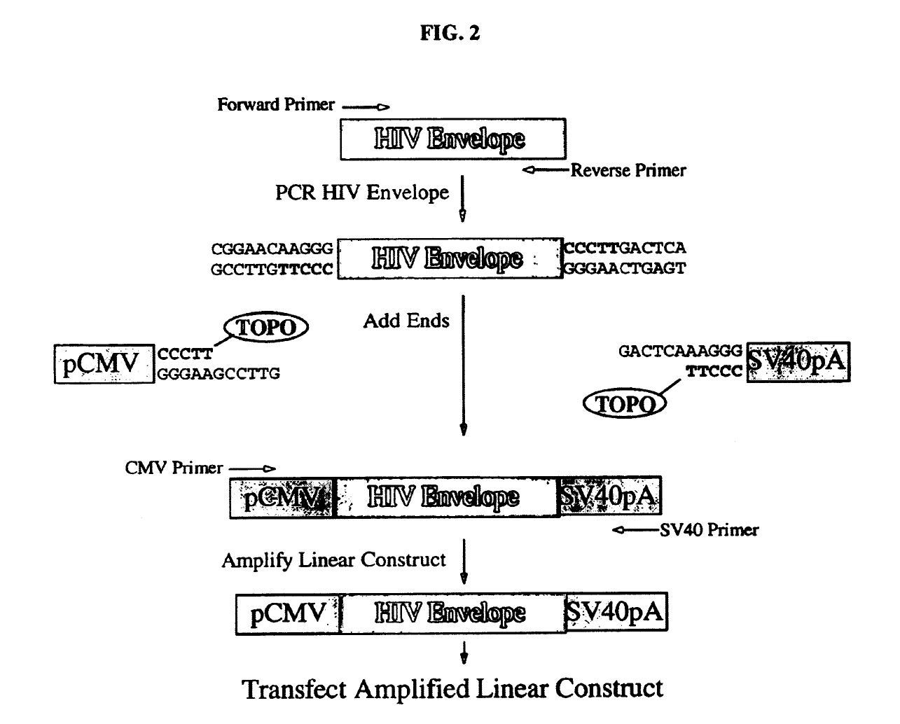

A 5′ promoter element and a 3′ polyadenylation element are attached to the viral envelope PCR fragment creating a viral envelope linear expression element as shown in FIG. 2. The attachment of the 5′ and 3′ elements can be carried out in about 10 minutes at room temperature. Preferably, the selected 5′ and 3′ sequences contain priming sites to conveniently perform secondary amplification of the linear viral envelope expression element. Examples of preferred 5′ and 3′ sequences are a 5′ CMV promoter element and a 3′ SV40 polyadenylation element.

The term “effector” as used herein refers to any cell, virus or other vehicle expressing a linear viral envelope expression element. The term “effector cell” as used herein refers to a host cell capable of expressing a linear viral envelope expression element and a transcription factor. In accordance with the present invention, the term “transcription factor” as used herein refers to a protein or peptide that is capable of activating transcription of a reporter element in the target cell.

In one embodiment of the present invention, effector cells are utilized which transiently express a linear viral envelope expression element and a transcription factor. In this embodiment, the linear envelope expression element is extrachromosomal. Alternatively, effector cells that are a stable cell line expressing linear viral envelope expression element integrated into a chromosome of the cells can be utilized.

In one embodiment of the present invention, the linear viral envelope expression element encodes a gp160 HIV envelope protein. In still another embodiment, a linear expression element containing a nucleic acid encoding a portion or variant of the HIV gp120 protein or a portion or variant of the HIV gp41 protein. For example, a linear viral envelope expression element including a nucleic acid encoding an HIV gp120 protein and the cytoplasmic domain of gp41 can be utilized. The HIV gp120 protein and the shortened gp41 are processed correctly and transported to the cell membrane. The effector cells for use in accordance with the present invention are capable of properly processing the HIV envelope glycoprotein including glycosylation, cleavage of gp160 precursor to gp120 and gp41 envelope proteins, and transport of the envelope proteins to the cell surface.

It will be understood by those skilled in the art that any conventional method for transfecting a nucleic acid construct into a host cell can be used to transfect the linear viral envelope expression element into a desired host cell. In a preferred embodiment of the present invention, the linear viral envelope expression element is cotransfected into the effector cell with a plasmid including a nucleic acid encoding the transcription factor. Alternatively, the viral linear envelope expression element and nucleic acid sequence encoding the transcription factor can be separately transfected into the effector cell. Examples of methods for transfecting the linear viral envelope expression element into a host cell are lipofection, electroporation, calcium phosphate and vaccinia virus infection. Preferably, lipofectamine is used to cotransfect the linear viral envelope expression element and the nucleic acid encoding the transcription factor into the effector cell.

The effector cells are preferably mammalian cells, more preferably, human cells. Examples of suitable host cells for use as effector cells are HeLa, 293T, B-SC-1 and NIH3T3 cells. In accordance with the most preferred embodiment of the present invention, the effector cells are HeLa cells.

In an alternative embodiment of the present invention, the nucleic acid sequence encoding the transcription factor can be included as part of the linear viral envelope expression element. It will be understood by those skilled in the art that the viral envelope linear expression element can include any other desired nucleic acid sequences in addition to the nucleic acid sequence encoding a viral envelope protein. However, the additional nucleotides in the viral envelope linear expression element is preferably limited to a number of nucleotides that does not prevent or impede amplification of the viral envelope linear expression element.

In accordance with the present invention, an inducible expression system can be used to induce expression of the reporter element of the target cell upon fusion of the effector cell and target cell. An example of a suitable expression system for use in the assay of the present invention is the Tet-Off Gene Expression System from BD Biosciences Clontech, Inc., Palo Alto, Calif. This system uses a constitutive promoter to express a transcription factor in the effector cell that activates transcription of the reporter element of the target cell. In a preferred embodiment of the present invention, the constitutive promoter is a CMV promoter and the transcription factor contains a tetracycline repressor protein. The transcription factor binds to a nucleic acid in the target cell resulting in expression of a reporter. Preferably, the transcription factor binds to a response element in the target cell resulting in expression of a reporter.

Examples of other promoters suitable for use in expressing the transcription factor are T7 promoter for use with transcription factor T7 polymerase, and HIV LTR for use with TAT transcription factor.

In the most preferred embodiment of the present invention, Hela H1 cells transiently expressing a HIV envelope linear expression element and the Tet-Off transcription factor are used as the effector cells.

As used herein, the term “target cell” refers to a cell (1) capable of fusing with an effector cell in the absence of a viral entry inhibitor and (2) containing a reporter. In a preferred embodiment of the present invention, the target cells contain a nucleic acid encoding a reporter and express one or more receptors necessary for fusion of the target cell with the effector cell. In a more preferred embodiment of the present invention, the target cell is capable of expressing a CD4 receptor and either CCR5 or CXCR4, or both CCR5 and CXCR4. Use of a target cell capable of expressing CCR5 and CXCR4 is especially preferable when the tropism of the HIV strain from a clinical sample is unknown. Alternatively, an effector cell and a target cell expressing a CD4 receptor and either a CCR5 receptor or a CXCR4 receptor can be utilized to determine the tropism of an HIV strain.

As used herein, the term receptor refers to a functional receptor protein and functional receptor protein variants including functional receptor proteins that are portions of a full length or wild type receptor protein.

In accordance with the present invention, the target cells contain a nucleic acid encoding a reporter under the control of a promoter capable of responding to the transcription factor of the effector cell. In a preferred embodiment of the present invention, the promoter is a Tet responsive element promoter (TRE) commercially available from BD Biosciences Clontech. Examples of suitable methods for detecting expression of the reporter include visually monitoring changes in morphology of the cell such as formation of syncytium by counting multinucleated cells in a microscopic field, evaluating fusion by fluorescent dye transfer, i.e., “redistribution/dequenching” assays, monitoring membrane lipid mixing and cytosolic content mixing. Particular examples of suitable reporters are beta-galactosidase (Nussbaum, O. et al., J. Virol., 68: 5411-5422(1994)) luciferase (Rucker, J. et al., Methods Enzymol., 288: 118-133 (1997)) chloramphenicol acetyltransferase, alkaline phosphatase, and green fluorescent protein and beta lactamase. In the most preferred embodiment of the present invention, the extent of cell fusion is monitored by chemiluminesence.

The target cells are preferably mammalian cells, and more preferably human cells. In one embodiment, target cells endogenously expressing human CD4 receptor are used in the assay of the present invention. Alternatively, a nucleic acid sequence encoding human CD4 can be introduced into the target cell by transfection such as for example using lipofectamine, vaccinia virus or other conventional method for introducing a nucleic acid encoding CD4 into the target cells. Moreover, the target cells of the present invention also express one or more endogenous or recombinant coreceptors, such as a chemokine receptor necessary for fusion of the viral envelope protein of interest to the target cell. Expression of CD4 and the chemokine receptor(s) must be sufficient to allow target cells to fuse with the effector cells. Examples of suitable promoters for use in expressing CD4 and/or a chemokine receptor are cytomegalovirus (CMV), and Rous sarcoma virus (RSV).

In accordance with one embodiment of the present invention, effector cells and target cells are separately incubated to allow for protein expression. The effector cells and target cells are then mixed and cultured under conditions that permit fusion of the effector cell with the target cell. Upon fusion of the effector cell and target cell, the transcription factor from the effector cell activates the reporter of the target cell. Preferably, the effector and target cells are incubated for about 10 to about 20 hours at 37° C. Following incubation of the effector and target cells the target cell reporter activity is measured.

In the most preferred embodiment of the present invention, the target cells are Hela H1 cells expressing CD4, CXCR4 and CCR5, and include a luciferase gene under the control of a TRE. In this embodiment, cell fusion occurs following the mixing of effector and target cells allowing the activation of the luciferase gene by the TET-Off transciption factor supplied by the effector cells. The extent of cell fusion is monitored by chemiluminescence of the luciferase protein. Any conventional method or device for measuring chemiluminescence of the luciferase protein can be used, for example detection of luciferase activity with a luminometer. The sensitivity of viral envelopes to viral entry inhibitors is determined by the addition of viral entry inhibitors during the cell fusion step.

Alternatively, the activity of the reporter can be evaluated by visually monitoring changes in morphology of the cell such as formation of syncytia. Visualization of syncytia can be accomplished by methods known to those skilled in the art such as fixing the target cells with formaldehyde/glutaraldehyde in PBS and washing and staining the cells with methylene blue and pararosanilinine in methanol.

The present invention provides a method of screening a sample for sensitivity to a viral entry inhibitor comprising the steps of:

(a) culturing in the presence of a viral entry inhibitor (i) an effector cell expressing a linear viral envelope expression element and a transcription factor with (ii) a target cell capable of fusing with the effector cell in the absence of the viral entry inhibitor, said target cell having a nucleic acid encoding a reporter wherein the nucleic acid is capable of expressing the reporter in response to the transcription factor of the effector cell; and

(b) detecting expression of the reporter wherein expression of the reporter is indicative of sensitivity of the sample to the viral entry inhibitor.

The assay of this invention is particularly useful for screening a sample for sensitivity to an HIV entry inhibitor and, more particularly, to an HIV-1 entry inhibitor. In a preferred embodiment, the linear viral envelope expression element comprises an HIV-1 viral envelope protein. Preferably, the target cells expresses CD4, CXCR4 and CCR5.

In another embodiment of the present invention, a method for monitoring a patient for sensitivity to a viral entry inhibitor is provided comprising the steps of:

(a) culturing in the presence of a viral entry inhibitor (i) a first effector cell expressing a linear viral envelope expression element prepared from a sample obtained from the patient at a first time and a transcription factor, with (ii) a first target cell capable of fusing with the first effector cell in the absence of the viral entry inhibitor, the first target cell containing a nucleic acid encoding a reporter wherein the nucleic acid is capable of expressing the reporter in response to the transcription factor of the first effector cell;

(b) culturing in the presence of the viral entry inhibitor (ii) a second effector cell expressing a linear viral envelope expression element prepared from a sample obtained from the patient at a second time, and a transcription factor with (ii) a second target cell capable of fusing with the second effector cell in the absence of the viral entry inhibitor, the second target cell containing a nucleic acid encoding a reporter wherein the nucleic acid is capable of expressing the reporter in response to the transcription factor of the second effector cell; and

(c) determining whether there is altered expression of the reporter in the second target cell when compared to the expression of the reporter in the first target cell wherein a change in expression of the reporter in the second target cell compared to the expression of the reporter in the first target cell indicates altered sensitivity of the patient to the viral entry inhibitor.

This invention provides an assay that is particularly useful for monitoring sensitivity of a patient to HIV entry inhibitors more particularly, to an HIV-1 entry inhibitor.

The present invention further provides a method for identifying viral entry inhibitors comprising the steps of:

(a) culturing in the presence of a candidate viral entry inhibitor (i) an effector cell expressing a linear viral envelope expression element and a transcription factor with (ii) a target cell capable of fusing with the effector cell in the absence of the viral entry inhibitor, the target cell containing a nucleic acid encoding a reporter wherein the nucleic acid is capable of expressing the reporter in response to the transcription factor of the effector cell; and

(b) determining whether there is a reduction in expression of the reporter in the presence of the candidate viral entry inhibitor when compared to the expression of the reporter in the absence of the candidate viral entry inhibitor wherein a reduction in expression of the reporter in the presence of the candidate viral entry inhibitor when compared to the expression of the reporter in the absence of the candidate viral entry inhibitor indicates that the candidate viral entry inhibitor is a viral entry inhibitor. In the most preferred embodiment of the method for screening for viral inhibitors, the target cell expresses CD4, CXCR4 and CCR5.

In an alternative embodiment of the invention, rather than utilizing an effector cell, the effector is a pseudovirus produced through cotransfection of a host cell with a linear viral envelope expression element and a modified viral genome plasmid to form a “production cell.” In this embodiment, host cells can be transfected with a population of linear viral envelope expression elements representative of the envelope population of a given clinical sample. The term “production cell” as used herein refers to a host cell (i) capable of expressing a linear viral envelope expression element and (ii) including a plasmid containing a modified viral genome. Preferably, the plasmid containing the modified viral genome contains a deletion of the envelope gene (ΔEnv) and incorporates a gene encoding a reporter. Examples of suitable reporters are firefly luciferase and renilla luciferase. More preferably, the linear viral envelope expression element encodes a gp160 HIV envelope protein and the production cell is capable of processing the HIV envelope protein through proper glycosylation and cleavage of gp160 to gp120 and gp41. In this embodiment, the linear envelope expression element and the modified viral genome plasmid are both extrachromosomal. Moreover, in this embodiment of the invention, the modified HIV genome plasmid is capable of being expressed and assembled into a viral capsid with its replication proteins. The viral capsid can assemble at the cell surface in association with a viral envelope containing region of the cell membrane and be released from the production cell as a virion particle. In accordance with the present invention, after cotransfecting the production cell with the linear viral envelope expression element and the modified viral genome plasmid, the production cell is incubated and the culture media becomes populated with pseudovirus particles. The be removed, clarified by centrifugation, and used directly or stored as a source of pseudovirus for subsequent infection of a host cell.

The production cells are preferably mammalian cells, and more preferably, human cells. Examples of suitable host cells for use as production cells are 293T, HeLa and NIH3T3 cells. In accordance with the most preferred embodiment of the present invention, the production cells are 293T cells.

In this embodiment, it is not necessary for the pseudovirus to include a transcription factor. However, if desired, the pseudovirus can include a transcription factor.

In this embodiment the target cell is capable (1) of fusing with the pseudovirus in the absence of a viral entry inhibitor and (2) expressing the reporter of the infecting pseudovirus. Preferably, the target cells are capable of expressing a CD4 receptor and either CCR5 or CXCR4 or both CCR5 and CXCR4. Use of a target cell capable of expressing CCR5 and CXCR4 is preferable when the tropism of the HIV strain from the clinical sample is unknown. Alternatively, a pseudovirus may be used separately with a target cell line expressing a CD4 receptor and either a CCR5 or a CXCR4 receptor to determine the tropism of an HIV strain. Peripheral blood mononuclear cells are suitable for use as target cells.

When utilizing a pseudovirus containing a reporter, it is preferable to utilize a target cell that does not include a reporter in order to reduce any background signal.

As described above, the pseudovirus can be used in a method of screening a sample for sensitivity to a viral entry inhibitor comprising the steps of:

(a) culturing in the presence of a viral entry inhibitor (i) a pseudovirus produced from the expression of a linear viral envelope expression element, wherein the pseudovirus includes a reporter, and (ii) a target cell capable of fusing with the pseudovirus in the absence of a viral entry inhibitor and expressing the reporter of the pseudovirus; and

(b) detecting expression of the reporter wherein expression of the reporter is indicative of sensitivity of the sample to the viral entry inhibitor. A pseudovirus can also be used in a method for monitoring a patient for sensitivity to a viral entry inhibitor comprising the steps of:

(a) culturing in the presence of a viral entry inhibitor (i) a first pseudovirus produced from the expression of a linear viral envelope expression element prepared from a sample obtained from the patient at a first time, wherein the pseudovirus includes a reporter, and (ii) a first target cell capable fusing with the pseudovirus in the absence of a viral entry inhibitor and expressing the reporter of the pseudovirus;

(b) culturing in the presence of the viral entry inhibitor (ii) a second pseudovirus produced from the expression of a linear viral expression element prepared from a sample obtained from the patient at a second time, wherein the second pseudovirus includes a reporter, and (ii) a second target cell capable of fusing with the pseudovirus in the absence of the viral entry inhibitor and expressing the reporter of the pseudovirus; and

(c) determining whether there is a change in expression of the reporter in the second target cell compared to the expression of the reporter in the first target cell wherein a change in expression of the reporter in the second target cell compared to the expression of the reporter in the first target cell indicates altered sensitivity of the patient to the viral entry inhibitor.

In accordance with the method for monitoring a patient for sensitivity to a viral entry inhibitor, an increase in expression of the reporter in the second target cell compared to expression of the reporter in the first target cell indicates decreased sensitivity of the patient to the viral entry inhibitor.

Moreover, pseudovirus can be used in a method for identifying viral entry inhibitors comprising the steps of:

(a) culturing in the presence of a candidate viral entry inhibitor (i) a pseudovirus produced from the expression of a linear viral envelope expression element, wherein the pseudovirus includes a reporter, and (ii) a target cell capable of fusing with the pseudovirus in the absence of a viral entry inhibitor and expressing the reporter of the pseudovirus; and

(b) determining whether there is a reduction in expression of the reporter in the presence of the candidate viral entry inhibitor when compared to the expression of the reporter in the absence of the candidate viral entry inhibitor wherein a reduction in expression of the reporter in the presence of the candidate viral entry inhibitor when compared to the expression of the reporter in the absence of the candidate viral entry inhibitor indicates that the candidate viral entry inhibitor is a viral entry inhibitor.

The method for identifying viral entry inhibitors provided herein is also particularly useful for determining if a known viral inhibitor is a viral entry inhibitor, i.e., inhibits fusion of the viral envelope protein to a host cell.

In another embodiment of the invention, a method of determining an inhibitory concentration of a viral entry inhibitor to inhibit virus infection in a patient is provided comprising the steps of:

(a) culturing in the presence of a concentration of viral entry inhibitor (i) an effector cell expressing a linear viral envelope expression element and a transcription factor with (ii) a target cell capable of fusing with the effector cell in the absence of the viral entry inhibitor, the target cell containing a nucleic acid encoding a reporter wherein the nucleic acid is capable of expressing the reporter in response to the transcription factor of the effector cell; and

(b) determining the concentration of viral entry inhibitor that inhibits a desired percentage of viral replication of the virus.

Preferably, the concentration of viral entry inhibitor that inhibits at least 50% of viral replication is determined.

Alternatively, the method for determining an inhibitory concentration of a viral entry inhibitor to inhibit virus infection in a patient comprises the steps of:

(a) culturing in the presence of a concentration of viral entry inhibitor (i) a pseudovirus produced from the expression of a linear viral envelope expression element, wherein the pseudovirus includes a reporter, and (ii) a target cell, wherein the target cell is capable of fusing with the pseudovirus and expressing the reporter element of the pseudovirus; and

(b) determining the concentration of viral entry inhibitor that inhibits a desired percentage of viral replication of the virus.

Preferably, the concentration of viral entry inhibitor that inhibits at least 50% of viral replication is determined.

In still another embodiment of the invention, the sensitivity of viral envelopes to entry inhibitors is determined by the addition of varying concentrations of viral entry inhibitor during the step of culturing effectors and target cells. A method of determining an inhibitory concentration of a viral entry inhibitor to inhibit virus infection in a patient is provided comprising the steps of:

(a) culturing in the presence of a first concentration of viral entry inhibitor (i) a first effector expressing a linear viral envelope expression element and a transcription factor with (ii) a first target cell capable of fusing with the first effector in the absence of the viral entry inhibitor, the target cell containing a nucleic acid encoding a reporter wherein the nucleic acid is capable of expressing the reporter in response to the transcription factor of the first effector; and

(b) culturing in the presence of a second concentration of viral entry inhibitor (i) a second effector expressing a linear viral envelope expression element and a transcription factor with (ii) a second target cell capable of fusing with the second effector in the absence of the viral entry inhibitor, the second target cell containing a nucleic acid encoding a reporter wherein the nucleic acid is capable of expressing the reporter in response to the transcription factor of the second effector; and

(c) determining the concentration of viral entry inhibitor that inhibits the desired percentage of viral replication of the virus. In accordance with the present invention, the method of determining an inhibitory concentration of a viral entry inhibitor to inhibit virus infection in a patient can be carried out using an effector that is a cell or a pseudovirus. As discussed above, rather than utilizing a target cell containing a reporter, a pseudovirus including a reporter and a target cell capable of expressing the reporter of the pseudovirus can be used.

In accordance with the present invention, the effective dose of viral entry inhibitor to be administered to a patient can be accurately and efficiently determined. Administration regimens of viral entry inhibitors can be tailored to a particular patient and modified as needed.

This invention further provides a method for evaluation the effectiveness of antiviral vaccines. In this embodiment, antibodies produced in response to the vaccine are added to the effector and target cells to evaluate the ability of the antibodies to inhibit fusion of the effector and target cells. The present invention provides a method for rapidly determining the effectiveness of antibodies to inhibit fusion of diverse viral envelope proteins with target cells.

In another embodiment, the present invention provides a method for monitoring viral fitness by monitoring fusion kinetics of the virus of interest. The fusion kinetics of viruses having variant envelope protein sequences can be determined by monitoring fusion of effector cells with target cells over a period of time, for example, for a period of from about 1 to about 12 hours. The fusion kinetics of mutant viral envelope sequences in the presence or absence of viral entry inhibitors can be readily determined using the effector cells and target cells described herein.

The assays described herein may be conducted as high-throughput assays. Techniques for performing high-throughput assays include use of microtiter-plates or pico-, nano- or micro-liter arrays. The assays of the invention are designed to permit high throughput screening of large compound libraries, e.g., by automating the assay steps and providing candidate viral entry inhibitors from any source to assay. Assays which are run in parallel on a solid support (e.g., microtiter formats on microtiter plates in robotic assays) are well known. Automated systems and methods for detecting and measuring changes in optical detection (or signal) are known. (See for example U.S. Pat. Nos. 6,171,780, 5,985,214, and 6,057,114).

In the assays of the invention, it is desirable to run positive controls to ensure that the components of the assays are working properly. It will also be appreciated that candidate viral entry inhibitors can be combined with other compounds having known effects on viruses. For example, known viral inhibitors can be used to find viral entry inhibitors that further effect the viral inhibition caused by the presence of the known viral inhibitor.

### EXAMPLES

The invention is illustrated by the following examples. These examples are illustrative only and do not limit the scope of the invention in any way.

### Example 1

Preparation of Linear HIV Envelope Expression Element HIV viral RNA was isolated from 140 μl culture supernatant using a QIAamp Viral RNA Mini Kit (Qiagen Inc). The viral RNA was eluted in 60 μl of elution buffer. First strand cDNA was synthesized using the SuperScript First-Strand Synthesis system for RT-PCR (Invitrogen Corporation) using 8 μl of viral RNA and random hexamers according to the manufactures instructions. The HIV envelope gene was PCR amplified using 4 μl of first strand CDNA, primers BF17 5′-ATGGAGCCAGTAGATCCTAGACTAGAGCCCTGG-3′ and ENV-N 5′-CTGCCAATCAGGGAAGTAGCCTTGTGT-3′ and a Platimun Taq DNA Polymerase High Fidelity Kit (Invitrogen) in a 100 μl reaction. The PCR product was purified using a QIAquick PCR Purification Kit (Qiagen Inc, Valencia, Calif.). In order to introduce the proper ends for the TOPO reaction 1 μl of the first PCR reaction was PCR amplified using primers:

BR1072
5′-CGGAACAAGGGCTTAGGCATCTCCTATGGCAGGAAGAA-3′ and
BR1075
5′-TGAGTCAAGGGTAGCCCTTCCAGTCCCCCCTTTTCTTTTA-3 or
CDIN5′
5′-CGGAACAAGGGATGGCAGGAAGAAGAAGCGGAGACAGC-3′ and
CDIN3′
5′-TGAGTCAAGGGTCTTATAGCAAAATCCTTTCCAAGCCCTGT-3′

in 100 μl as before. The PCR reaction was run on a 0.7% agarose gel and the about 3 kb HIV envelope gene was gel purified using a QIAquick Gel Extraction KIT (Qiagen Inc). The 5′ CMV promoter element and 3′ SV40 polyadenylation element were added to 700 ng of the HIV envelope PCR fragment using a TOPO Tools Kit (Invitrogen) and the final PCR product was purified as before.

### Example 2

Preparation of Effector Cells

The effector cells were prepared by transfecting 1.7×106 Hela H1 cells in a T75 flask with the linear viral envelope expression element and pTET-Off using a Lipofectamine Plus Kit (Invitrogen) as follows. 3 μg of the linear envelope expression vector and 3 μg of pTET-OFF were mixed with 750 μl of D-MEM media. 49 μl of Plus Reagent was added to the DNA mixture, the solution was mixed. After 15 minutes at room temperature a solution consisting of 750 μl D-MEM and 30 μl of lipofectamine was added to the DNA solution. The solution was mixed and incubated at room temperature for 15 minutes. The media from 1.7×106 Hela H1 cells ion a T75 flask was removed, the cells were washed with 15 ml of D-PBS, and 6 ml of D-MEM without serum was added to the cells. The DNA/lipofectamine solution was mixed, added to the cells and the cells were then incubated at 37° C. in the presence of 5% CO2. After 4 hours, the transfection mixture was removed and replaced with 15 ml of D-MEM 10% FBS and incubated overnight at 37° C. in the presence of 5% CO2.

### Example 3

Preparation of Target Cells Hela H1 cells expressing CD4, CXCR4, CCR5, and containing an integrated copy of a TRE/luciferase reporter gene were used as target cells. These cells were maintained in DMEM containing 10% FBS, 0.2 mg/ml G418, 0.4 mg/ml zeocin, 0.1 mg/ml hygromycin B, and 1.5 μg/ml puromycin.

### Example 4

Cell Mediated Fusion Assay

The target cells and effector cells were removed from their flasks after 24 hours incubation at 37° C. in the presence of 5% CO2 using trypsin. The effector and target cells were mixed at a ratio of 1:2, respectively, and seeded in a 96 well plate at 5×104 cells per well in the presence of various concentrations of the test compound. After 12 to 18 hours incubation at 37° C. in the presence of 5 % CO2 the media was removed from the cells, the luciferase activity was determined using a Steady-Glo Luciferase Assay System (Promega Corp., Madison, Wis.) and the EC50 was determined.

### Example 5

Comparison of Assays Using Linear Viral Envelope Expression Elements vs. Whole Virus

To demonstrate that this assay can be used to determine the sensitivity of HIV envelopes to HIV entry inhibitors, two laboratory strains of HIV, LAI and NL4-3, were used as a source of HIV envelope. Linear HIV-LAI envelope expression elements were prepared using the TOPO Tools Technique which were then used in the cell mediated fusion assay to determine the EC50 to an HIV entry inhibitor BMS-806 (Lin P-F. et al., Identification and Characterization of a Novel Inhibitor of HIV-1 Entry- I: Virology and Resistance, 9th Conference on Retroviruses and Opportunistic Infections, Seattle, Washington (2002) and Lin P-F. et al., Identification and Characterization of a Novel Inhibitor of HIV-1 Entry - II: Mechanism of Action, 9th Conference on Retroviruses and Opportunistic Infections, Seattle, Wash. (2002)) (Table 1). The data demonstrate that the EC50 values generated using the HIV envelope linear expression element are similar to the EC50 values obtained with whole HIV viruses according to a method as described in Lin, P-F. et al., Antimicrobial Agents and Chemotherapy, Vol. 40: 133-138 (1996).

TABLE 1
Comparison of cell fusion and whole Virus EC50s

BMS-806 EC50 (nM)

| | TOPO Cell | |
|---|---|---|
| Envelope | Fusion | Whole Virus |
| NL4-3 | 5.7 | 2.1 |
| LAI | 3.5 | 2.5 |

### Example 6

Comparison of Assays Using Clinical Linear Viral Envelope Expression Element vs. Whole Virus

To demonstrate that this assay can be used to determine the sensitivity of HIV envelopes derived from HIV infected patients to HIV entry inhibitors, HIV infected serum samples and HIV infected PBMCs were used as a source of HIV envelope. Again linear HIV envelope expression elements were prepared using the TOPO Tools Technique which were then used in the cell mediated fusion assay to determine the sensitivity of the envelopes to BMS-806 (Table 2). Again, the EC50 values generated using the HIV envelope linear expression element are similar to the EC50 values obtained with the whole HIV virus.

TABLE 2
EC50 Values for Clinical Envelopes

BMS-806 EC50 (nM)

| | | TOPO | |
|---|---|---|---|
| Envelope | Source | Fusion | Whole Virus |
| BRH04820 | Serum | 5.5 | 5.5 |
| BRH04821 | Serum | 2.7 | 5.3 |
| 92US072 | PBMC | 818 | 336 |
| 93US157 | PBMC | 2.3 | 3.9 |

### Example 7

Preparation of Pseudovirus in 293T Production Cells Pseudovirus is prepared by co-transfecting 2×10⁶ 293T cells in a T75 flask with the (i) linear viral envelope expression element, (ii) pTET-Off and (iii) ΔEnv Bru Luc (modified viral genome plasmid) using a Lipofectamine Plus Kit (Invitrogen) as follows. 6.5 μg of the linear envelope expression vector, 1.5 μg of pTET-OFF, and 6.5 μg ΔEnv Bru Luc are mixed with 650 μl of Opti Mem I serum free medium (Invitrogen). 22 μl of Plus Reagent is gradually added to the DNA mixture, with careful mixing to prevent DNA precipitation. After 15 minutes at room temperature a solution consisting of 650 μl Opti Mem I and 50 μl of lipofectamine is added to the DNA solution. The solution is mixed and incubated at room temperature for 15 minutes, after which the DNA mixture is brought to 7.5 ml with Opti Mem I. The media from the T75 flask of 293T cells are removed, and the DNA mixture is added, without washing. The cells are incubated at 37° C. in the presence of 5 % C02 for 3-4 hours, after which the transfection mixture is removed and replaced with 25 ml of D-MEM 10% FBS. The flask is taken to a P3 biocontainment lab, where incubation at 37° C. in the presence of 5% CO2, is continued for 3 days. At this point the pseudovirus-containing supernatant is removed aseptically, clarified in a clinical centrifuge, and aliquoted for storage at -80° C.

### Example 8

Pseudovirus Infection Assay

The target cells Hela 67 cells (CD4+CCR5+CXCR4+) are maintained in D-MEM +10% FBS, containing the selection antibiotics zeocin (400 μg/ml), G-418 (200 μg/ml) and hygromycin (100 μg/ml). One day prior to the assay, the cells are washed and trypsinized, and then diluted to 10,000 cells/ml in D-MEM +10% FBS. 100 μl aliquots are dispensed into individual wells of a 96-well, flat bottom tissue culture dish. The plate is incubated overnight at 37° C. in the presence of 5% CO2 to allow all the cells to adhere. On day 2, test compound (2 μl) is added to each well. Test compounds are typically prepared at 100-fold the final concentration in a vehicle of dimethylsulfoxide. Immediately afterward, 100 μl of prewarmed pseudovirus solution is added. The pseudovirus is typically diluted 2-4 fold just prior to addition, using fresh DMEM +10% FBS. After three days of incubation at 37° C. in the presence of 5% CO2 the media is removed from the cells, and the luciferase activity is determined using a Firefly Luciferase Assay System, as described by the manufacturer (Roche Molecular Biochemicals). The EC50 is determined, using an XL-fit program.

It will be apparent that certain changes and modifications may be practiced within the scope of the appended claims.

What is claimed is:

1. A method of screening a sample for sensitivity to a viral entry inhibitor comprising the steps of:
(a) culturing in the presence of a viral entry inhibitor (i) an effector expressing a linear viral envelope expression element and a transcription factor with (ii) a target cell capable of fusing with the effector in the absence of the viral entry inhibitor, said target cell containing a nucleic acid encoding a reporter wherein said nucleic acid is capable of expressing the reporter in response to the transcription factor of the effector; and
(b) detecting expression of the reporter wherein expression of the reporter is indicative of sensitivity of the sample to the viral entry inhibitor.

2. The method of claim 1 wherein the viral entry inhibitor is an HIV entry inhibitor.

3. The method of claim 2 wherein the viral entry inhibitor is an HIV-1 entry inhibitor.

4. The method of claim 1 wherein the effector is a cell.

5. The method of claim 4 wherein the effector is a mammalian cell.

6. The method of claim 5 wherein the effector is a human cell.

7. The method of claim 1 wherein the effector is a pseudovirus.

8. The method of claim 1 wherein the target cell is a mammalian cell.

9. The method of claim 8 wherein the target cell is a human cell.

10. The method of claim 8 wherein the target cell expresses a CD4 receptor.

11. The method of claim 8 wherein the target cell expresses a receptor selected from the group consisting of CXCR4, CCR5, CCR2b and CCR3.

12. The method of claim 1 wherein the linear viral envelope expression element comprises an HIV viral envelope protein.

13. The method of claim 1 wherein the reporter is selected from the group consisting of beta-galactosidase, luciferase, chloramphenicol acetyltransferase, alkaline phosphatase, green fluorescent protein and beta lactamase.

14. A method for monitoring a patient for sensitivity to a viral entry inhibitor comprising the steps of:
(a) culturing in the presence of a viral entry inhibitor (i) a first effector expressing a linear viral envelope expression element prepared from a sample obtained from the patient at a first time and a transcription factor with (ii) a first target cell capable of fusing with the first effector in the absence of the viral entry inhibitor, said first target cell containing a nucleic acid encoding a reporter wherein said nucleic acid is capable of expressing the reporter in response to the transcription factor of the first effector; and
(b) culturing in the presence of the viral entry inhibitor (i) a second effector expressing a linear viral envelope expression element prepared from a sample obtained from the patient at a second time and a transcription factor with (ii) a second target cell capable of fusing with the second effector in the absence of the viral entry inhibitor, said second target cell containing a nucleic acid encoding a reporter wherein said nucleic acid is capable of expressing the reporter in response to the transcription factor of the second effector; and
(c) determining whether there is a change in expression of the reporter in the second target cell when compared to the expression of the reporter in the first target cell wherein an increase in expression of the reporter in the second target cell compared to the expression of the reporter in the first target cell indicates decreased sensitivity of the patient to the viral entry inhibitor.

15. The method of claim 14 wherein the viral entry inhibitor is an HIV entry inhibitor.

16. The method of claim 15 wherein the viral entry inhibitor is an HIV-1 entry inhibitor.

17. The method of claim 14 wherein the effector cell is a mammalian cell.

18. The method of claim 17 wherein the effector cell is a human cell.

19. The method of claim 14 wherein the target cell is a mammalian cell.

20. The method of claim 19 wherein the target cell is a human cell.

21. The method of claim 14 wherein the linear viral envelope expression element comprises an HIV viral envelope protein.

22. The method of claim 14 wherein the target cell expresses a CD4 receptor

23. The method of claim 14 wherein the target cell expresses a receptor selected from the group consisting of CXCR4, CCR5, CCR2b and CCR3.

24. The method of claim 14 wherein the reporter is selected from the group consisting of beta-galactosidase, luciferase, chloramphenicol acetyltransferase, alkaline phosphatase, green fluorescent protein and beta lactamase.

25. A method for identifying viral entry inhibitors comprising the steps of:
(a) culturing in the presence of a candidate viral entry inhibitor (i) an effector cell expressing a linear viral envelope expression element and a transcription factor with (ii) a target cell capable of fusing with the effector cell in the absence of the viral entry inhibitor, said target cell containing a nucleic acid encoding a reporter wherein said nucleic acid is capable of expressing the reporter in response to the transcription factor of the effector cell; and
(b) determining whether there is a reduction in expression of the reporter in the presence of the candidate viral entry inhibitor when compared to the expression of the reporter in the absence of the candidate viral entry inhibitor wherein a reduction in expression of the reporter in the presence of the candidate viral entry inhibitor when compared to the expression of the reporter in the absence of the candidate viral entry inhibitor indicates that the candidate viral entry inhibitor is a viral entry inhibitor.

26. The method of claim 25 wherein the target cell expresses a CD4 receptor

27. The method of claim 25 wherein the target cell expresses a receptor selected from the group consisting of CXCR4, CCR5, CCR2b and CCR3.

28. A method of determining an inhibitory concentration of a viral entry inhibitor to inhibit virus infection in a patient comprising the steps of:
(a) culturing in the presence of a concentration of viral entry inhibitor (i) an effector expressing a linear viral envelope expression element and a transcription factor with (ii) a target cell capable of fusing with the effector in the absence of the viral entry inhibitor, the target cell containing a nucleic acid encoding a reporter wherein the nucleic acid is capable of expressing the reporter in response to the transcription factor of the effector; and
(b) determining the concentration of viral entry inhibitor that inhibits a desired percentage of viral replication of the virus.

29. The method of claim 28, wherein step (b) comprises determining the concentration of viral entry inhibitor that inhibits at least 50% of viral replication of the virus.

30. The method of claim 28, wherein the effector is a cell.

31. The method of claim 28, wherein the effector is a pseudovirus.

32. A method of screening a sample for sensitivity to a viral entry inhibitor comprising the steps of:
(a) culturing in the presence of a viral entry inhibitor (i) a pseudovirus produced from the expression of a linear viral envelope expression element, wherein the pseudovirus includes a reporter, and (ii) a target cell capable of fusing with the pseudovirus in the absence of the viral entry inhibitor, wherein the target cell is capable of expressing the reporter of the pseudovirus; and
(b) detecting expression of the reporter wherein expression of the reporter is indicative of sensitivity of the sample to the viral entry inhibitor.

33. The method of claim 32 wherein the viral entry inhibitor is an HIV entry inhibitor.

34. The method of claim 33 wherein the viral entry inhibitor is an HIV-1 entry inhibitor.

35. The method of claim 32 wherein the target cell is a mammalian cell.

36. The method of claim 35 wherein the target cell is a human cell.

37. The method of claim 32 wherein the target cell expresses a CD4 receptor.

38. The method of claim 32 wherein the target cell expresses a receptor selected from the group consisting of CXCR4, CCR5, CCR2b and CCR3.

39. The method of claim 32 wherein the linear viral envelope expression element comprises an HIV viral envelope protein.

40. The method of claim 32 wherein the reporter is selected from the group consisting of beta-galactosidase, luciferase, chloramphenicol acetyltransferase, alkaline phosphatase, green fluorescent protein and beta lactamase.

41. A method for identifying viral entry inhibitors comprising the steps of:
(a) culturing in the presence of a candidate viral entry inhibitor (i) a pseudovirus produced from the expression of a linear viral envelope expression element, wherein the pseudovirus includes a reporter, and (ii) a target cell capable of fusing with the pseudovirus in the absence of the viral entry inhibitor, wherein the target cell is capable of expressing the reporter of the pseudovirus; and
(b) determining whether there is a reduction in expression of the reporter in the presence of the candidate viral entry inhibitor when compared to the expression of the reporter in the absence of the candidate viral entry inhibitor wherein a reduction in expression of the reporter in the presence of the candidate viral entry inhibitor when compared to the expression of the reporter in the absence of the candidate viral entry inhibitor indicates that the candidate viral entry inhibitor is a viral entry inhibitor.

42. The method of claim 41 wherein the viral entry inhibitor is an HIV entry inhibitor.

43. The method of claim 41 wherein the target cell expresses a CD4 receptor

44. The method of claim 41 wherein the target cell expresses a receptor selected from the group consisting of CXCR4, CCR5, CCR2b and CCR3.

45. The method of claim 41 wherein the linear viral envelope expression element comprises an HIV viral envelope protein.


SEQUENCE LISTING (9 sequences)

SEQ ID NO: 1 - DNA, 33 bp, Artificial
  Primers
atggagccagtagatcctagactagagccctgg

SEQ ID NO: 2 - DNA, 27 bp, Artificial
  Primers
ctgccaatcagggaagtagccttgtgt

SEQ ID NO: 3 - DNA, 11 bp, Artificial
  Primers
cggaacaaggg

SEQ ID NO: 4 - DNA, 11 bp, Artificial
  Primers
tgagtcaaggg

SEQ ID NO: 5 - DNA, 38 bp, Artificial
  Primers
cggaacaagggcttaggcatctcctatggcaggaagaa

SEQ ID NO: 6 - DNA, 40 bp, Artificial
  Primers
tgagtcaagggtagcccttccagtccccccttttctttta

SEQ ID NO: 7 - DNA, 38 bp, Artificial
  Primers
cggaacaagggatggcaggaagaagaagcggagacagc

SEQ ID NO: 8 - DNA, 41 bp, Artificial
  Primers
tgagtcaagggtcttatagcaaaatcctttccaagccctgt

SEQ ID NO: 9 - DNA, 33 bp, Artificial; identical to SEQ ID NO: 1
  Primers